(19) United States
(12) Patent Application Publication
(10) Pub. No.: US 2020/0370090 A1
(43) Pub. Date: Nov. 26, 2020

(54) COMPOSITIONS AND METHODS FOR DETECTING ALLERGENS

(71) Applicant: KIMBBERLY-CLARK WORLDWIDE, INC., Neenah, WI (US)

(72) Inventors: John Gavin MACDONALD, Decatur, GA (US); Waihong LEONG, Alpharetta, GA (US)

(73) Assignee: Kimberly-Clark Worldwide, Inc., Neenah, WI (US)

(21) Appl. No.: 16/634,239

(22) PCT Filed: Jul. 31, 2017
(86) PCT No.: PCT/US17/44587

§ 371 Date: Jan. 27, 2020

(51) Int. Cl. C12Q 1/37; G01N 21/78

(52) U.S. Cl. CPC C12Q 1/37; G01N 21/78; G01N 2333/8142; G01N 2333/8139; G01N 2800/24

(57) ABSTRACT

Compositions and methods for detecting food-based allergens on a surface are disclosed. A composition can include ninhydrin and a protease enzyme. The protease enzyme can be a cysteine protease enzyme, an aspartic protease enzyme, or combinations thereof. In some embodiments, the composition can include a carrier. Methods of detecting a food-based allergen on a surface can include providing a composition including ninhydrin and providing a protease enzyme. The method can include applying the composition and the protease enzyme to the surface. The method can also include allowing the ninhydrin and the protease enzyme to interact with particulates from the surface for a detection period. Detecting the food-based allergen can occur when the composition changes color after the detection period.

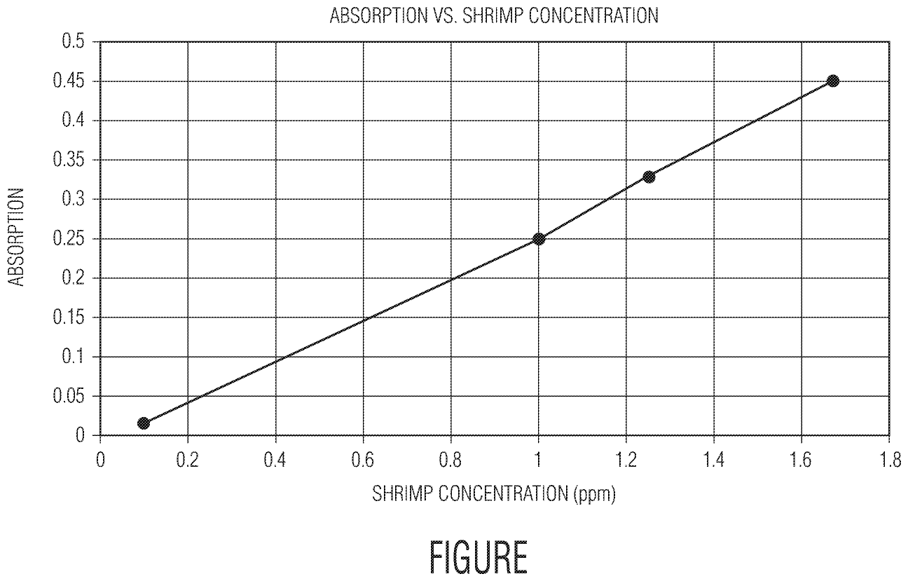

### TECHNICAL FIELD

The present disclosure relates to compositions and methods for detecting allergens. More specifically, the present disclosure relates to compositions and methods for detecting food-based allergens.

### BACKGROUND OF THE DISCLOSURE

Allergic reactions to certain food types or ingredients is becoming a large issue with a significant percentage of the population. As an example, peanut (Arachis hypogaea L.) allergy is amongst the most important food allergies in western societies, not only because of high incidence rate in populations, but also because of the severity of its associated symptoms. Allergens are proteins characterized by their ability to induce a pathogenic immunoglobulin E (IgE) response in susceptible individuals. Both genetic and environmental factors can influence susceptibility.

The handling of foods or ingredients including allergens is a big challenge to commercial food preparation companies, which typically use their machines to make a variety of foods. Recently, food manufacturers have been under increased regulation to ensure proper and thorough cleaning is performed on their machines. Food-based allergens can also present issues in kitchens, restaurants, and on other food-preparatory or handling surfaces in various environments. When cleaning surfaces, such as food processing equipment or food preparatory surfaces, it is unclear if the surface is sufficiently clean of the food particulates including the allergens. In some circumstances, having remaining particulates including allergens, even in trace amounts, could present a significant issue. For example, trace amounts of allergens remaining on a surface could contaminate the future processing or handling of the next batch of food or item. This is critically important in commercial food processing equipment which is used for a variety of different types of food that can include allergens, such as, but not limited to, peanuts, wheat, or shell fish containing products.

While one can attempt to clean a surface (e.g., a preparatory table or equipment), it is difficult to know whether such surface is sufficiently clean and free from allergens. Thus, there remains a need to allow a user to quickly see if there are allergens present on a surface. If allergens are present, there also remains a need to provide an indication of where such allergens remain so they can be further cleaned before further processing or preparatory work is undertaken.

### SUMMARY OF THE DISCLOSURE

In one embodiment, a composition for detecting a food-based allergen on a surface is provided. The composition can include ninhydrin. The composition can also include a protease enzyme selected from the group consisting of: a cysteine protease enzyme, an aspartic protease enzyme, and combinations thereof.

In another embodiment, a method of detecting a food-based allergen on a surface is provided. The method can include providing a composition comprising ninhydrin. The method can also include providing a protease enzyme selected from the group consisting of: a cysteine protease enzyme, an aspartic protease enzyme, and combinations thereof. The method can additionally include applying the composition and the protease enzyme to the surface. The method can also include allowing the ninhydrin and the protease enzyme to interact with particulates from the surface for a detection period. The method can include detecting the food-based allergen when the composition changes color after the detection period.

### BRIEF DESCRIPTION OF DRAWINGS

The FIGURE illustrates a graph of Absorption vs. Shrimp Concentration for Example 11 discussed herein.

### DETAILED DESCRIPTION OF THE DISLOSURE

The present disclosure is directed to compositions and methods for detecting allergens. In particular, the disclosure is directed to compositions including ninhydrin and a cysteine protease enzyme or an aspartic protease enzyme (or combinations thereof) and methods of using such a composition to detect food-based allergens. The compositions can be used to detect the presence of a food-based allergen on a surface. As used herein, a “surface” can be any abiotic or biotic surface. Various exemplary surfaces in which the composition be applied to detect the presence of an allergen include, but are not limited to: a food preparatory countertop, food processing equipment, food service table, various foods, and skin.

Most allergens in foods are small proteins or peptides. Ninhydrin (2, 2-dihydroxyindane-1,3-dione), which can be obtained from Sigma-Aldrich Chemical Company, Milwaukee Wis., reacts covalently with the amines of various protein-based allergens at ambient temperature to develop a visible purple color to indicate the presence of an allergen (in the Examples herein, the color of the starting composition was colorless). In doing so, the composition can also potentially inactivate protein-based allergen's allergenic activity. The table below lists the top eight allergenic foods and the allergens within these foods.

TABLE 1
Eight common allergenic foods and associated allergens

| Allergenic Food | Allergen within the food |
|---|---|
| Peanuts | Protein Epitopes (Ara h 1-7) - protein |
| Crustacean shellfish | Alpha-Tropomyosin - protein |
| (e.g., shrimp) | |
| Egg | egg white: ovomucoid, ovalbumin, |
| | ovotransferrin, and lysozyme; ovomucoid, |
| | also called Gal d 1protein |
| | egg yolk: livetin, apovitillin, and phosvitin |
| Fish | Parvalbumin β - protein |
| Milk | alpha S1 -casein, a protein |
| Soy | B3 polypeptide-protein |
| Tree nuts | Amandin; 14S globulin ~400K Dalton - protein |
| Wheat | Pro-Ser-Gin-Gin and Gin-Gin-Gin-Pro Peptides |

In the compositions of the present disclosure, embodiments can include ninhydrin and either a cysteine protease enzyme or an aspartic protease enzyme (or combinations thereof). Various other components could also form part of the composition, however, are not necessary and could be removed in some embodiments. Suitable optional ingredients include, but are not limited to, carriers, humectants, emollients, surfactants, emulsifiers, builders, sequestrants, chelators, preservatives, pH modifiers, preservatives/antimicrobial agents, disinfectants, colorants, rheology modifiers, antioxidants, anti-parasitic agents, antipruritics, antifungals, antiseptic actives, astringents, keratolytic actives, local anaesthetics, anti-stinging agents, anti-reddening agents, skin soothing agents, external analgesics, film formers, sunscreens, deodorants, fragrance, and various other optional ingredients as are known by one skilled in the art.

Ninhydrin can form from about 0.1% to about 50.0% of the composition (by total weight of the composition). In some preferred embodiments, ninhydrin can form from about 5.0% to about 20.0% of the composition (by total weight of the composition). Of course, some compositions can have ninhydrin outside of these preferred ranges and still be within the scope of the disclosure. Ninhydrin reacts covalently with amines and amino groups, and therefore, is believed to inactivate the allergen molecules rendering them no longer allergically active. This provides an additional benefit to the composition other than signifying the presence of an allergen.

As noted above, the composition can also include a protease enzyme that is either a cysteine protease enzyme or an aspartic protease enzyme. In combination with ninhydrin, cysteine protease enzymes and aspartic protease enzymes can significantly reduce the length of the detection period in which the composition changes color to signify the presence of a food-based allergen (as will be discussed further in the Examples below), yet surprisingly do not change the color of the composition including the ninhydrin by themselves. Reducing the time to detection can be beneficial for a user looking to detect if an allergen is present, and if so, where further cleaning may be required. It was discovered that not all protease enzymes could be utilized with ninhydrin in the composition because some protease enzymes react with ninhydrin itself to change the color of the composition, even when not being placed in contact with an allergen (see Example 13). Thus, it was discovered that only cysteine and aspartic protease enzymes can be used with ninhydrin in the allergen detecting composition of the present disclosure to avoid providing false positives of allergens on a surface.

In some embodiments, the ninhydrin and the protease enzyme can be kept separate from one another (either in separate compositions or as separate components) and applied separately to the desired surface. However, it is preferred that the ninhydrin and the protease enzyme form a part of the same composition that can be applied at the same time to the desired surface.

In some embodiments, the protease enzyme can comprise about 0.01% to about 20.0% of the composition (by total weight of the composition), and preferably more preferably from about 0.05% to about 10.0% of the composition (by total weight of the composition). In some embodiments, a ratio of the ninhydrin to the protease enzyme can be from about 200:1 to about 1:1, and more preferably, from about 150:1 to about 50:1. It is believed that by having a higher amount of protease enzyme in the composition can aid the composition in changing color more quickly, and thus, provide an earlier indication of the presence of an allergen. Of course, some compositions can have amounts of protease enzymes and ratios of ninhydrin to the protease enzyme outside of these preferred ranges and still be within the scope of the disclosure.

Compositions of the present disclosure may be formulated with one or more conventional and compatible carrier materials. Liquid carrier materials suitable for use in the instant disclosure include those well-known for use in the cosmetic, pharmaceutical, and medical arts as a basis for ointments, lotions, creams, salves, aerosols, gels, suspensions, sprays, foams, washes, and the like, and may be used in their established levels. In some embodiments, the carrier can comprise from about 0.01% to about 99.0% (by total weight of the composition), depending on the carrier used, and more preferably, from about 10.0 to about 90% (by total weight of the composition).

Preferable carrier materials include alcohols or water/alcohol solvent materials, such as ethanol or isopropyl alcohol or water alcohol mixtures. Other potential carriers include water, emollients, humectants, polyols, surfactants, esters, perfluorocarbons, silicones, and other pharmaceutically acceptable carrier materials. In one embodiment, the carrier is volatile, allowing for immediate deposition of the composition to the desired surface while reducing drying time. Non-limiting examples of these volatile carriers include 5 cst Dimethicone, Cyclomethicone, Methyl Perfluoroisobutyl Ether, Methyl Perfluorobutyl Ether, Ethyl Perfluoroisobutyl Ether and Ethyl Perfluorobutyl Ether. In some embodiments, the ratio between the carrier and the protease enzyme can be from about 1:1 to about 1000:1 (by total weight of the composition), more preferably from about 10:1 to about 900:1 (by total weight of the composition), and even more preferably from about 100:1 to about 850:1 (by total weight of the composition). In some preferred embodiments, the ratio of the carrier to the protease enzyme can be about 800:1 (by total weight of the composition). Of course, some compositions can have ratios outside of these preferred ranges and still be within the scope of the disclosure.

The compositions of the present disclosure could be used in various forms including, but not limited to, in a solution form that could be applied to a surface by pouring, by spraying, or via a wet wipe or roll-on applicator. If used in a spray application, the composition could be sprayed by equipment known to those of ordinary skill in the art, such as a mechanical or battery-operated spray bottle, aerosol spray, or other suitable mechanism. The composition could also be in the form of a dry coating that could be applied to a substrate, including, but not limited to: a wipe, paper towel, glove, swab, head to foot PPE, hair net, face mask, air filter (e.g., KCPs EnFresh* or Entrepid*), or foot covers. In some embodiments, the composition could be in liquid form and applied to a substrate, such as a wipe substrate, an absorbent substrate, a fabric or cloth substrate, a tissue or paper towel substrate, or the like. In one embodiment, the composition may be used in combination with a wipe substrate to form a wet wipe or may be a wetting composition for use in combination with a wipe which may be dispersible. In other embodiments, the antimicrobial composition may be incorporated into wipes such as wet wipes, hand wipes, face wipes, cosmetic wipes, cloths and the like.

In one embodiment, the wet wipe may comprise a nonwoven material that is wetted with an aqueous solution termed the “wetting composition,” which may include or be composed entirely of the compositions disclosed herein. As used herein, the nonwoven material comprises a fibrous material or substrate, where the fibrous material or substrate comprises a sheet that has a structure of individual fibers or filaments randomly arranged in a mat-like fashion. Nonwoven materials may be made from a variety of processes including, but not limited to, airlaid processes, wet-laid processes such as with cellulosic-based tissues or towels, hydroentangling processes, staple fiber carding and bonding, melt blown, and solution spinning.

The fibers forming the fibrous material may be made from a variety of materials including natural fibers, synthetic fibers, and combinations thereof. The choice of fibers may depend upon, for example, the intended end use of the finished substrate and the fiber cost. For instance, suitable fibers may include, but are not limited to, natural fibers such as cotton, linen, jute, hemp, wool, wood pulp, etc. Similarly, suitable fibers may also include: regenerated cellulosic fibers, such as viscose rayon and cuprammonium rayon; modified cellulosic fibers, such as cellulose acetate; or synthetic fibers, such as those derived from polypropylenes, polyethylenes, polyolefins, polyesters, polyamides, polyacrylics, etc. Regenerated cellulose fibers, as briefly discussed above, include rayon in all its varieties as well as other fibers derived from viscose or chemically modified cellulose, including regenerated cellulose and solvent-spun cellulose, such as Lyocell. Among wood pulp fibers, any known papermaking fibers may be used, including softwood and hardwood fibers. Fibers, for example, may be chemically pulped or mechanically pulped, bleached or unbleached, virgin or recycled, high yield or low yield, and the like. Chemically treated natural cellulosic fibers may be used, such as mercerized pulps, chemically stiffened or crosslinked fibers, or sulfonated fibers.

In addition, cellulose produced by microbes and other cellulosic derivatives may be used. As used herein, the term “cellulosic” is meant to include any material having cellulose as a major constituent, and, specifically, comprising at least 50 percent by weight cellulose or a cellulose derivative. Thus, the term includes cotton, typical wood pulps, non-woody cellulosic fibers, cellulose acetate, cellulose triacetate, rayon, thermomechanical wood pulp, chemical wood pulp, debonded chemical wood pulp, milkweed, or bacterial cellulose. Blends of one or more of any of the previously described fibers may also be used, if so desired.

The fibrous material may be formed from a single layer or multiple layers. In the case of multiple layers, the layers are generally positioned in a juxtaposed or surface-to-surface relationship and all or a portion of the layers may be bound to adjacent layers. The fibrous material may also be formed from a plurality of separate fibrous materials wherein each of the separate fibrous materials may be formed from a different type of fiber.

Airlaid nonwoven fabrics are particularly well suited for use as wet wipes. The basis weights for airlaid nonwoven fabrics may range from about 20 to about 200 grams per square meter (gsm) with staple fibers having a denier of about 0.5 to about 10 and a length of about 6 to about 15 millimeters. Wet wipes may generally have a fiber density of about 0.025 g/cc to about 0.2 g/cc. Wet wipes may generally have a basis weight of about 20 gsm to about 150 gsm. More desirably the basis weight may be from about 30 to about 90 gsm. Even more desirably the basis weight may be from about 50 gsm to about 75 gsm.

Processes for producing airlaid non-woven basesheets are described in, for example, published U.S. Pat. App. No. 2006/0008621, herein incorporated by reference to the extent it is consistent herewith.

In some embodiments when the composition is used as a wetting composition with a substrate, the wetting composition can be applied to the substrate at an add-on percentage of from about 30% to about 500%, or from about 125% to about 400%, or from about 150% to about 350%.

Various methods of using the compositions as discussed herein to detect a food-based allergen on a surface can be utilized. A method can include providing a composition including ninhydrin and either a cysteine protease enzyme or an aspartic protease enzyme. The method can include applying the composition to the surface. The method can include allowing the composition to interact with particulates from the surface for a detection period. As used herein, “detection period” is a period of time required for the composition to change color noticeable to the human eye to indicate the presence of a food-based allergen. As such, the method can include detecting the food-based allergen when the composition changes color after the detection period. In some embodiments, it is preferable to have a composition that can provide a detection period less than five minutes, or more preferably, less than three minutes.

As noted above, the composition can interact with particulates from the surface in various ways. For example, the composition can be applied to the surface (e.g., by spraying or pouring) and then the composition can be allowed to interact with particulates from the surface by remaining on the surface itself. Alternatively, the composition could be on a substrate, and the composition interact with particulates from the surface by wiping the surface with the substrate. In such a situation, the composition can interact with the particulates form the surface on the substrate. As discussed above, various substrates could be utilized, including wipes, towels, gloves, etc.

### EXAMPLES

The following examples provide exemplary compositions and methods for detecting food-based allergens. In the examples, various particulates including known allergens were spread on surfaces and exemplary compositions were sprayed onto the surfaces including the particulates to determine if the compositions could effectively detect the allergens.

### EXAMPLE 1

Samples of freshly ground peanuts, peanut butter (Allergenic epitopes, Jiff Creamy, J. M. Smucker Company, Orrville, Ohio), fresh shrimp (Alpha-tropomyosin allergen, Georgia gulf fresh shrimp, Publix), and wheat flour (Pilsbury Best All Purpose flour, J. M. Smucker Company, Orrville, Ohio) were streaked onto sheets of writing paper. This was done by taking a small amount of each (50 mg) on a gloved fingertip and rubbing the finger down the paper to leave a 4″×0.5″ streak of each food on the paper. These streaks were then lightly sprayed with a solution of 2.0 g of Ninhydrin (Sigma-Aldrich Chemical Company, Milwaukee Wis.) in 20 ml of reagent grade ethanol (Sigma-Aldrich) using a sprayer available from Prevail Sprayer, Prevail Company, Coal City, Ill. After one minute at ambient temperature (21° C.) the composition in the area of the shrimp streak started to turn a visible purple in color followed by the composition in the area of the ground peanuts and the composition in the area of the peanut butter after five minutes. The composition in the area of the wheat flower did not show signs of any purple color even after thirty minutes.

### EXAMPLE 2

Example 1 was repeated but this time the paper was placed onto a warm (39° C.) hot plate before being sprayed with the ninhydrin solution as described in Example 1. After light spraying the composition in the area of the shrimp streak turned purple in color after one minute followed by the composition in the area of the ground peanuts and the composition in the area of the peanut butter in three minutes and the composition in the area of the wheat flour in five minutes. Clearly, this slight warming help to speed up the color development of the composition.

### EXAMPLE 3

Example 1 was repeated but this time the streaks were sprayed with a solution containing mixture of 1.0 g of ninhydrin in 10.0 g ethanol and 1.2 g meat tenderizer containing papain, a cysteine protease of the peptidase C1 family. It is believed that the meat tenderizer included papain at an amount of between 0.5-10% wt/wt of the meat tenderizer, and more likely between 0.5-2.0% wt/wt. Papain consists of a single polypeptide chain with three disulfide bridges and a sulfhydryl group necessary for the activity of the enzyme. The meat tenderizer was manufactured by The Kroger Company, Cincinnati, Ohio, and was placed in 7 ml of saline solution added together and stirred with the ninhydrin and ethanol solution. The solution was lightly sprayed onto the streaks on paper and observed at ambient temperature. The composition in the area of the shrimp turned purple in color in under one minute followed by the composition in the area of the ground peanuts and the composition in the area of the peanut butter in three minutes followed by the wheat flour in five minutes. Clearly the use of a proteinase enzyme helped to speed up the color development.

### EXAMPLE 4

Example 3 was repeated, but this time the paper containing the food streaks was placed onto a hotplate at 39° C. before spraying with the composition of Example 3 that included the ninhydrin and meat tenderizer having papain. After light spraying, the composition in the area of the shrimp streak turned purple in under thirty seconds followed by the composition in the area of the ground peanuts and the composition in the area of the peanut butter in under two minutes followed by the composition in the area of the wheat flour in three minutes. From this example, it appears that the gentle warming seems to have further increased the color development kinetics of the composition. In fact, after ten minutes the purple color of the composition had turned almost to a purple/black in intensity.

### EXAMPLE 5

Example 4 was repeated but this time the food streaks were done on a steel plate (12″×3″×⅛″ thickness). The steel plate was placed on a hot plate set at 39° C. before spraying. Similar results and timing was observed. The purple color was still easily observed even on the steel plate.

### EXAMPLE 6

Example 6 provided an experiment to determine if the ninhydrin would react with the cysteine protease enzyme of papain. To do so, the composition of Example 3 (1.0 g of ninhydrin in 10.0 g ethanol and 1.2 g meat tenderizer containing papain) was sprayed onto a sheet of paper and left overnight at ambient temperature. No color developed during that time. From this result, it was established that ninhydrin does not react with the papain enzyme to develop the purple color in the composition.

### EXAMPLE 7

Fresh whole milk (Lactose allergen) was streaked onto a sheet of paper by dipping a gloved fingertip into the liquid milk and then dragging the wetted finger down a sheet of paper to yield a 2″ by 2″ patch. This was lightly sprayed with the indicator mixture used in Example 3 (1.0 g of ninhydrin in 10.0 g ethanol and 1.2 g meat tenderizer containing papain). Within three minutes, a purple color was observed to form in the area the composition was applied, and which continued to deepen in color over time.

### EXAMPLE 8

A small part of a snack bar containing soy (Banana Honey Nut Breakfast Flats, Quaker Oats Company, Chicago Ill.) was crumbled and the fragments (e.g., particulates) rubbed down a sheet of paper. This area was then lightly sprayed with the indicator mixture used in Example 3 (1.0 g of ninhydrin in 10.0 g ethanol and 1.2 g meat tenderizer containing papain). Within three minutes, the area of the composition applied to the smear and crumbs started to turn visually purple, with the color darkening further after five minutes.

### EXAMPLE 9

Fresh egg whites were streaked onto a sheet of paper by dipping a gloved fingertip into the liquid egg whites and then dragging the wetted finger down a sheet of paper to yield a 4″ by 1″ patch. The area of the egg whites was lightly sprayed with the ninhydrin indicator solution described in Example 1 (2.0g of Ninhydrin in 20 ml of reagent grade ethanol). Within three minutes, a very visible purple color could be seen in the area of the composition, which continued to deepen in color over time.

### EXAMPLE 10

Example 1 was repeated but this time clams (wild caught, Bumble Bee Company, San Diego, Calif.) were used and tested as the particulates. The liquid from the can of minced clams was streaked down a sheet of paper using a gloved finger to yield a 4″×0.5″ streak. Next, the solution of ninhydrin indicator was sprayed on the streak. Within three minutes, the purple color was clearly visible in the area of the composition that was sprayed on the paper near the liquid from the can of minced claims. This further extends the application of this novel indicator. Shrimp are a member of crustacean shellfish and clams are a member of molluscan shell fish. Both groups turn purple with this indicator composition.

### EXAMPLE 11

In order to determine how sensitive the indicator composition is to allergenic foods a standard curve was measured using shrimp, and is illustrated in the FIGURE. One gram of pulverized shrimp was dispersed in one liter of deionized water. Then, one milliliter of this solution was dissolved into each of 600 ml, 800 ml, and 1000 ml of deionized water, respectively. Also, 10 ml of the 1000 ml shrimp solution was then dissolved into 100 ml. 1 ml (5% wt/wt) of the ninhydrin indicator in ethanol solution was added to each of the above diluted solutions and the absorption reading measured at 575 nm using a UV/Vis instrument (Thermo Scientific Evolution 220 Ultraviolet-Visible Spectrum spectrophotometer). Table 2 shows the shrimp concentration (ppm) and the Absorption as measured by the UV/Vis instrument.

TABLE 2
Shrimp concentration (ppm) vs. Absorption data

| Shrimp Concentration (ppm) | Absorption |
|---|---|
| 1.67 | 0.45 |
| 1.25 | 0.33 |
| 1.00 | 0.25 |
| 0.01 | 0.015 |

The FIGURE shows that a straight line standard curve was obtained by graphing the data points of Table 2. As a result, in some embodiments, the composition of the present disclosure can be effective at detecting a protein-based allergen down to 0.01 ppm.

### EXAMPLE 12

Two allergenic items that the ninhydrin based indicator composition did not detect included latex (natural rubber) and sulfites. In testing against the composition of Example 3 (1.0 g of ninhydrin in 10.0 g ethanol and 1.2 g meat tenderizer containing papain), the composition did not change color. This is not too surprising since neither are protein-based because there are no amino groups in their chemistries. Latex is cis-polyisoprene (a hydrocarbon, ˜38K Dalton) and sulfites (sodium bisulfite) is an inorganic salt. Both the latex and the sulfites were obtained from Sigma-Aldrich Chemical Company, Milwaukee, Wis.

### EXAMPLE 13

In addition to the cysteine protease enzyme of papain, three additional types of protease enzymes were tested for their use in conjunction with ninhydrin in an allergen detection composition to see if they would trigger a color change in the composition without being in the presence of an allergen.

TABLE 3
List of various protease groups and enzymes tested with Ninhydrin

| | | Reacts with Ninhydrin to |
|---|---|---|
| Enzyme Group Type | Enzyme Used | form color? |
| Metalloprotease | Thermolysin | Yes |
| Aspartic protease | Cathepsin | No |
| Serine protease | Trypsin | Yes |
| Cysteine protease | Papain | No |

As shown in Table 3, the aspartic protease and cysteine proteases do not react with ninhydrin to cause a change in color in the composition, and thus, are suitable for use in an allergen detection composition. However, surprisingly, the enzyme groups of metalloprotease (thermolysin) and serine protease (trypsin) reacted with the ninhydrin to cause a color change, without being in the presence of an allergen. Based on this reaction, these enzyme groups would not be suitable for use in an allergen detection composition because they could lead to potential false positive detection of allergens.

### Embodiments

Embodiment 1: A composition for detecting a food-based allergen on a surface, the composition comprising: ninhydrin; and a protease enzyme selected from the group consisting of: a cysteine protease enzyme, an aspartic protease enzyme, and combinations thereof.

Embodiment 2: The composition of embodiment 1, further comprising a carrier.

Embodiment 3: The composition of embodiment 2, wherein the carrier is ethanol.

Embodiment 4: The composition of embodiment 2 or 3, wherein a ratio of the carrier to the protease enzyme is between 10:1 to 900:1.

Embodiment 5: The composition of any one of the preceding embodiments, wherein the protease enzyme is a cysteine protease enzyme.

Embodiment 6: The composition of embodiment 5, wherein the protease enzyme is papain.

Embodiment 7: The composition of any one of embodiments 1-4, wherein the protease enzyme is an aspartic protease enzyme.

Embodiment 8: The composition of embodiment 7, wherein the protease enzyme is cathepsin.

Embodiment 9: The composition of any one of the preceding embodiments, wherein a ratio of the ninhydrin to the protease enzyme is between 1:1 to 200:1.

Embodiment 10: A method of detecting a food-based allergen on a surface, the method comprising: providing a composition comprising ninhydrin; providing a protease enzyme selected from the group consisting of: a cysteine protease enzyme, an aspartic protease enzyme, and combinations thereof; applying the composition and the protease enzyme to the surface; allowing the ninhydrin and the protease enzyme to interact with particulates from the surface for a detection period; and detecting the food-based allergen when the composition changes color after the detection period.

Embodiment 11: The method of embodiment 10, wherein the composition comprises the protease enzyme.

Embodiment 12: The method of embodiment 10 or 11, wherein the composition further comprises a carrier.

Embodiment 13: The method of embodiment 12, wherein the composition is applied to the surface by spraying, and wherein allowing the ninhydrin and the protease enzyme to interact with particulates from the surface for a detection period comprises allowing the composition to remain on the surface for at least the detection period.

Embodiment 14: The method of embodiment 12, wherein the composition is on a substrate, and wherein allowing the composition to interact with particulates from the surface for a detection period comprises wiping the surface with the substrate.

Embodiment 15: The method of embodiment 14, wherein the substrate is selected from the group consisting of: a glove, wipe, napkin, swab, dip stick, probe, machinery part, face mask, air filter, PPE, foot covering, and stretch wrap, and wherein the composition forms a coating on the substrate.

Embodiment 16: The method of any one of embodiments 10-15, wherein the detection period is less than five minutes.

Embodiment 17: The method of any one of embodiments 10-16, wherein the protease enzyme is a cysteine protease enzyme.

Embodiment 18: The method of embodiment 17, wherein the protease enzyme is papain.

Embodiment 19: The method of any one of embodiments 10-16, wherein the protease enzyme is an aspartic protease enzyme.

Embodiment 20: The method of embodiment 19, wherein the protease enzyme is cathepsin.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

What is claimed is:

1. A composition for detecting a food-based allergen on a surface, the composition comprising:
ninhydrin; and
a protease enzyme selected from the group consisting of: a cysteine protease enzyme, an aspartic protease enzyme, and combinations thereof.

2. The composition of claim 1, further comprising a carrier.

3. The composition of claim 2, wherein the carrier is ethanol.

4. The composition of claim 2, wherein a ratio of the carrier to the protease enzyme is between 10:1 to 900:1.

5. The composition of claim 1, wherein the protease enzyme is a cysteine protease enzyme.

6. The composition of claim 5, wherein the protease enzyme is papain.

7. The composition of claim 1, wherein the protease enzyme is an aspartic protease enzyme.

8. The composition of claim 7, wherein the protease enzyme is cathepsin.

9. The composition of claim 1, wherein a ratio of the ninhydrin to the protease enzyme is between 1:1 to 200:1.

10. A method of detecting a food-based allergen on a surface, the method comprising:
providing a composition comprising ninhydrin;
providing a protease enzyme selected from the group consisting of: a cysteine protease enzyme, an aspartic protease enzyme, and combinations thereof;
applying the composition and the protease enzyme to the surface;
allowing the ninhydrin and the protease enzyme to interact with particulates from the surface for a detection period; and
detecting the food-based allergen when the composition changes color after the detection period.

11. The method of claim 10, wherein the composition comprises the protease enzyme.

12. The method of claim 11, wherein the composition further comprises a carrier.

13. The method of claim 12, wherein the composition is applied to the surface by spraying, and wherein allowing the ninhydrin and the protease enzyme to interact with particulates from the surface for a detection period comprises allowing the composition to remain on the surface for at least the detection period.

14. The method of claim 12, wherein the composition is on a substrate, and wherein allowing the composition to interact with particulates from the surface for a detection period comprises wiping the surface with the substrate.

15. The method of claim 14, wherein the substrate is selected from the group consisting of: a glove, wipe, napkin, swab, dip stick, probe, machinery part, face mask, air filter, PPE, foot covering, and stretch wrap, and wherein the composition forms a coating on the substrate.

16. The method of claim 10, wherein the detection period is less than five minutes.

17. The method of claim 10, wherein the protease enzyme is a cysteine protease enzyme.

18. The method of claim 17, wherein the protease enzyme is papain.

19. The method of claim 10, wherein the protease enzyme is an aspartic protease enzyme.

20. The method of claim 19, wherein the protease enzyme is cathepsin.
